(19) United States
(12) Patent Application Publication
(10) Pub. No.: US 2003/0100580 A1
(43) Pub. Date: May 29, 2003

(54) Urotensin-II receptor antagonists

(76) Inventors: Dashyant Dhanak, West Chester, PA (US); Steven David Knight, West Chester, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/149,794

(22) PCT Filed: Dec. 19, 2000
(86) PCT No.: PCT/US00/34574

(51) Int. Cl. A61K 31/4709; A61K 31/47; A61K 31/4545; A61K 31/454; A61K 31/4535; A61K 31/4439; A61K 31/4025; A61K 31/433; A61K 31/4245; A61K 31/18

(52) U.S. Cl. 514/312; 514/345; 514/362; 514/365; 514/424; 514/317; 514/322; 514/326; 514/422; 514/441; 514/471; 514/408; 514/602; 546/153; 546/194; 546/199; 546/208; 546/212; 546/214; 546/232; 548/126; 548/527; 548/517; 548/570; 549/65; 549/475

(57) ABSTRACT

P51084 The present invention relates to sulfonamides, pharmaceutical compositions containing them, and their use as antagonists of urotensin II.

### FIELD OF THE INVENTION

The present invention relates to sulfonamides, pharmaceutical compositions containing them and their use as urotensin II antagonists

### BACKGROUND OF THE INVENTION

The integrated control of cardiovascular homeostasis is achieved through a combination of both direct neuronal control and systemic neurohormonal activation. Although the resultant release of both contractile and relaxant factors is normally under stringent regulation, an aberration in this status quo can result in cardiohemodynamic dysfunction with pathological consequences.

The principal mammalian vasoactive factors that comprise this neurohumoral axis, namely angiotensin-II, endothelin-1, norepinephrine, all function via an interaction with specific G-protein coupled receptors (GPCR). Urotensin-II, represents a novel member of this neurohumoral axis.

In the fish, this peptide has significant hemodynamic and endocrine actions in diverse end-organ systems and tissues:

smooth muscle contraction

both vascular and non-vascular in origin including smooth muscle preparations from the gastrointestinal tract, respiratory, and genitourinary tract. Both pressor and depressor activity has been described upon systemic administration of exogenous peptide

osmoregulation:

effects which include the modulation of transepithelial ion (Na⁺, Cl⁻) transport. Although a diuretic effect has been described, such an effect is postulated to be secondary to direct renovascular effects (elevated GFR)

metabolism:

urotensin-II influences prolactin secretion and exhibits a lipolytic effect in fish (activating triacylglycerol lipase resulting in the mobilization of non-esterified free fatty acids)

(Pearson, et. al. Proc. Natl. Acad. Sci. (U.S.A.) 1980, 77, 5021; Conlon, et. al. J. Exp. Zool. 1996, 275, 226.)

In studies with human Urotensin-II it was found that it:

was an extremely potent and efficacious vasoconstrictor

exhibited sustained contractile activity that was extremely resistant to wash out

had detrimental effects on cardiac performance (myocardial contractility)

Human Urotensin-II was assessed for contractile activity in the rat-isolated aorta and was shown to be the most potent contractile agonist identified to date. Based on the in vitro pharmacology and in vivo hemodynamic profile of human Urotensin-II it plays a pathological role in cardiovascular diseases characterized by excessive or abnormal vasoconstriction and myocardial dysfunction. (Ames et. al. Nature 1999, 401, 282).

Compounds that antagonize the Urotensin-II receptor may be useful in the treatment of congestive heart failure, stroke, ischemic heart disease (angina, myocardial ischemia), cardiac arrhythmia, hypertension (essential and pulmonary), COPD, restenosis, asthma, (Hay D W P, Luttmann M A, Douglas S A: 2000, Br J Pharmacol: volume 131, pages 10-12) neurogenic inflammation and metabolic vasculopathies all of which are characterized by abnormal vasoconstriction and/or myocardial dysfunction. Since U-II and GPR14 are both expressed within the mammalian CNS (Ames et. al. Nature 1999, 401, 282), they also may be useful in the treatment of addiction, schizophrenia, impulsivity, anxiety, stress, depression, and neuromuscular function. Functional U-II receptors are expressed in rhabdomyosarcomas cell lines and therefore may have oncological indications. Urotensin may also be implicated in various metabolic diseases such as diabetes (Ames et. al. Nature 1999, 401, 282, Nothacker et al., Nature Cell Biology 1: 383-385, 1999)

### SUMMARY OF THE INVENTION

In one aspect this invention provides for sulfonamides and pharmaceutical compositions containing them.

In a second aspect, this invention provides for the use of sulfonamides as antagonists of urotensin II, and as inhibitors of urotensin II.

In another aspect, this invention provides for the use of sulfonamides for treating conditions associated with urotensin II imbalance.

In yet another aspect, this invention provides for the use of sulfonamides for the treatment of congestive heart failure, stroke, ischemic heart disease (angina, myocardial ischemia), cardiac arrhythmia, hypertension (essential and pulmonary), COPD, restenosis, asthma, neurogenic inflammation and metabolic vasculopathies, addiction, schizophrenia, impulsivity, anxiety, stress, depression, neuromuscular function, and diabetes.

The urotensin antagonist may be administered alone or in conjunction with one or more other therapeutic agents, said agents being selected from the group consisting of endothelin receptor antagonists, angiotensin converting enzyme (ACE) inhibitors, vasopeptidase inhibitors, diuretics, digoxin, and dual non-selective β-adrenoceptor and α₁-adrenoceptor antagonists.

Other aspects and advantages of the present invention are described further in the following detailed description of the preferred embodiments thereof.

### DETAILED DESCRIPTION OF THE INVENTION

The present invention provides for compounds of Formula (I):

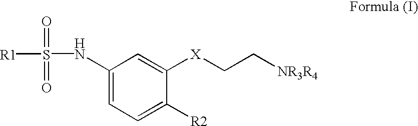

wherein:

R₁ is phenyl, benzothiophenyl, thienyl, furyl, pyrrolyl, pyridinyl, benzthiadiazoyl, benzoxadiazoyl, quinolinyl, or naphthyl, all of which may be substituted or unsubstituted by one, two, three, four or five of the following: halogen, methoxy, OH, NO₂, YCF₃, C₁₋₄ alkyl, C₍₀₋₄₎alkylCO₂C₍₀₋₄₎alkyl, cyano, CycloC₍₁₋₄₎alkylenedioxy, or dimethylamino;

R₂ is halogen, CN or methyl;

R₃ and R₄ are independently hydrogen, C₁₋₆ alkyl or benzyl; or with the nitrogen form a pyrrolidine or piperidine ring;

X is O or CH₂;

Y is a bond or O;

provided the compound of Formula (1) is not 5-Chloro-3-methyl-benzo[b]thiophene-2-sulfonic acid [3-(2-dimethylamino-ethoxy)-4-iodo-phenyl]-amide; or a pharmaceutically acceptable salt thereof.

When used herein, the term “alkyl” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the terms ‘halogen’ and ‘halo’ include fluorine, chlorine, bromine and iodine and fluoro, chloro, bromo and iodo, respectively.

The compounds of the present invention may contain one or more asymmetric carbon atoms and may exist in racemic and optically active form. All of these compounds and their diastereoisomers are contemplated to be within the scope of the present invention.

Preferably R₁ is phenyl, thienyl, pyridinyl, benzthiadiazoyl, benzoxadiazoyl, or naphthyl, all of which may be substituted or unsubstituted by one, two, or three of the following:

halogen, methoxy, NO₂, YCF₃, or C₁₋₄ alkyl.

Preferably R₂ is halogen.

Preferably R₃ is alkyl; more preferably R₃ is methyl or ethyl.

Preferably R₄ is alkyl; more preferably R₄ is methyl or ethyl.

Preferably X is O.

Preferably Y is a bond.

Preferred Compounds are:

N-[4-Iodo-3-(2-dimethylamino-ethoxy)-phenyl]-3,4-dimethoxy-benzenesulfonamide;

4-Bromo-N-[4-iodo-3-(2-dimethylamino-ethoxy)-phenyl]-benzenesulfonamide;

N-[4-Methyl-3-(2-dimethylamino-ethoxy)-phenyl]-3 ,4-dimethoxy-benzenesulfonamide;

N-[4-Iodo-3-(2-dimethylamino-ethoxy)-phenyl]-3-methoxy-benzenesulfonamide;

N-[4-Bromo-3-(2-dimethylamino-ethoxy)-phenyl]-3,4-dimethoxy-benzenesulfonamide;

N-[4-Chloro-3-(2-dimethylamino-ethoxy)-phenyl]-3,4-dimethoxy-benzenesulfonamide;

4,5-Dibromo-N-[4-chloro-3-(2-

dimethylamino-ethoxy)-phenyl]-2-thiophenesulfonamide;

3,4-Dibromo-N-[4-chloro-3-(2-dimethylamino-ethoxy)-phenyl]-benzenesulfonamide;

2,4,6-Trichloro-N-[4-chloro-3-(2-dimethylamino-ethoxy)-phenyl]-benzenesulfonamide;

2,6-Dichloro-N-[4-chloro-3-(2-dimethylamino-ethoxy)-phenyl]-4-trifluoromethyl-benzenesulfonamide;

2-Bromo-N-[4-chloro-3-(2-dimethylamino-ethoxy)-phenyl]-4,S-dimethoxy-benzenesulfonamide;

4-Bromo-N-[4-chloro-3-(2-dimethylamino-ethoxy)-phenyl]-benzenesulfonamide;

4-Iodo-N-[4-iodo-3-(2-dimethylamino-ethoxy)-phenyl]-benzenesulfonamide;

3,5-Dichloro-N-[4-iodo-3-(2-dimethylamino-ethoxy)-phenyl]-benzenesulfonamide;

2,3-Dichloro-N-[4-chloro-3-(2-dimethylamino-ethoxy)-phenyl]-benzenesulfonamide;

3-Chloro-4-fluoro-N-[4-chloro-3-(2-dimethylamino-ethoxy)-phenyl]-benzenesulfonamide;

3-Chloro-4-methyl-N-[4-chloro-3-(2-dimethylamino-ethoxy)-phenyl]-benzenesulfonamide;

2,5-Dimethyl-4-chloro-N-[4-chloro-3-(2-dimethylamino-ethoxy)-phenyl]-benzenesulfonamide;

2-Chloro-4-trifluoromethyl-N-[4-chloro-3-(2-dimethylamino-ethoxy)-phenyl]-benzenesulfonamide;

2,4-Dichloro-6-methyl-N-[4-iodo-3-(2-dimethylamino-ethoxy)-phenyl]-benzenesulfonamide;

3-Methoxy-N-[4-iodo-3-(2-dimethylamino-ethoxy)-phenyl]-benzenesulfonamide;

2,5-Dimethoxy-N-[4-chloro-3-(2-dimethylamino-ethoxy)-phenyl]-benzenesulfonamide;

2,5-Dimethoxy-N-[4-bromo-3-(2-dimethylamino-ethoxy)-phenyl]-benzenesulfonamide;

3-Nitro-N-[4-iodo-3-(2-dimethylamino-ethoxy)-phenyl]-benzenesulfonamide;

2-Nitro-4-methoxy-N-[4-iodo-3-(2-dimethylamino-ethoxy)-phenyl]-benzenesulfonamide;

3-Nitro-4-methyl-N-[4-chloro-3-(2-dimethylamino-ethoxy)-phenyl]-benzenesulfonamide;

2-Ethyl-4-bromo-N-[4-chloro-3-(2-dimethylamino-ethoxy)-phenyl]-benzenesulfonamide;

3,4-Dichlorophenyl-N-[4-chloro-3-(2-dimethylamino-ethoxy)-phenyl]-benzenesulfonamide;

2,4,6-Trimethyl-N-[4-chloro-3-(2-dimethylamino-ethoxy)-phenyl]-benzenesulfonamide;

4-Chloro-N-[4-chloro-3-(2-dimethylamino-ethoxy)-phenyl]-2-naphthalenesulfonamide;

5-Chloro-N-[4-chloro-3-(2-dimethylamino-ethoxy)-phenyl]-2-thiophenesulfonamide;

5-Dichloro-N-[4-iodo-3-(2-dimethylamino-ethoxy)-phenyl]-3-thiophenesulfonamide;

5-Bromo-N-[4-chloro-3-(2-dimethylamino-ethoxy)-phenyl]-2-thiophenesulfonamide;

4,5-Dichloro-N-[4-chloro-3-(2-dimethylamino-ethoxy)-phenyl]-2-thlophenesulfonamide;

5-({[1-(4-Chloro-phenyl)-methanoyl]-amino}methyl)-N-[4-chloro-3-(2-dimethylaminoethoxy)-phenyl]-2-thiophenesulfonamide;

N-[4-Iodo-3-(2-dimethylamino-ethoxy)-phenyl]-benzo[1 ,2,5]-4-thiadiazolesulfonamide;

2,4-Dichloro-N-[4-chloro-3-(2-dimethylamino-ethoxy)-phenyl]-benzenesulfonamide;

2-Methyl-4-bromo-N-[4-chloro-3-(2-dimethylamino-ethoxy)-phenyl]-benzenesulfonamide;

2,6-Dimethyl-4-bromo-N-[4-chloro-3-(2-dimethylamino-ethoxy)-phenyl]-benzenesulfonamide;

3-Methoxy-4-bromo-N-[4-chloro-3-(2-dimethylamino-ethoxy)-phenyl]-benzenesulfonamide;

2,4-Dichloro-5-methyl-N-[4-chloro-3-(2-dimethylamino-ethoxy)-phenyl]-benzenesulfonamide;

3-Nitro-4-chloro-N-[4-chloro-3-(2-dimethylamino-ethoxy)-phenyl]-benzenesulfonamide;

2-Nitro-4-trifluoromethyl-N-[4-chloro-3-(2-dimethylamino-ethoxy)-phenyl]-benzenesulfonamide;

5-Chloro-N-[4-chloro-3-(2-dimethylamino-ethoxy)-phenyl]-1-naphthalenesulfonamide;

4-Bromo-5-chloro-N-[4-chloro-3-(2-dimethylamino-ethoxy)-phenyl]-2-thiophenesulfonamide;

3-Bromo-5-chloro-N-[4-chloro-3-(2-dimethylamino-ethoxy)-phenyl]-2-thiophenesulfonamide;

4-Nitro-5-chloro-N-[4-chloro-3-(2-dimethylamino-ethoxy)-phenyl]-2-thiophenesulfonamide;

4,5-Dichloro-N-[4-iodo-3-(2-dimethylamino-ethoxy)-phenyl]-2-thiophenesulfonamide;

7-Chloro-N-[4-iodo-3-(2-dimethylamino-ethoxy)-phenyl]-benzo[1 ,2,5]oxadiazole-4-sulfonamide;

5-Bromo-6-chloro-N-[4-chloro-3-(2-dimethylamino-ethoxy)-phenyl]-3-pyridinesulfonamide;

2,4-Dibromo-5-methoxy-N-[4-chloro-3-(2-dimethylamino-ethoxy)-phenyl]-benzenesulfonamide;

2-Methyl-4,5-dimethoxy-N-[4-chloro-3-(2-dimethylamino-ethoxy)-phenyl]-benzenesulfonamide;

2,6-Dimethyl-4-bromo-N-[4-chloro-3-(2-diethylamino-ethoxy)-phenyl]-benzenesulfonamide;

3,4-Dimethoxy-N-[4-chloro-3-(2-methylamino-ethoxy)-phenyl]-benzenesulfonamide;

2-Bromo-4,5-dimethoxy-N-[4-chloro-3-(2-methylamino-ethoxy)-phenyl]-benzenesulfonamide;

5-Chloro-N-[4-chloro-3-(2-dimethylamino-ethoxy)-phenyl]-2-naphthalenesulfonamide;

2,6-Dichloro-4-trifluoromethoxy-N-[4-chloro-3-(2-diethylamino-ethoxy) -phenyl]-benzenesulfonamide;

4,5-Dibromo-N-[4-chloro-3-(2-diethylamino-ethoxy)-phenyl]-2-thiophenesulfonamide;

2-Bromo-4,5-dimethoxy-N-[4-chloro-3-(2-diethylamino-ethoxy)-phenyl]-benzenesulfonamide;

3,4-Dimethoxy-N-[4-chloro-3-(3-dimethylamino-propyl)-phenyl]-benzenesulfonamide;

3,4-Dimethoxy-N-[4-chloro-3-(2-diethylamino-ethoxy)-phenyl]-benzenesulfonamide;

2-Chloro-4,5-dimethoxy-N-[4-chloro-3-(2-diethylamino-ethoxy)-phenyl]-benzenesulfonamide; and

2-Chloro-4,5-dimethoxy-N-[4-chloro-3-(2-dimethylamino-ethoxy)-phenyl]-benzenesulfonamide.

More preferred compounds are:

N-[4-Chloro-3-(2-dimethylamino-ethoxy)-phenyl]-3,4-dimethoxy-benzenesulfonamide;

4,5-Dibromo-N-[4-chloro-3-(2-dimethylamino-ethoxy)-phenyl]-2-thiophenesulfonamide;

3,4-Dibromo-N-[4-chloro-3-(2-dimethylamino-ethoxy)-phenyl]-benzenesulfonamide;

2,4,6-Trichloro-N-[4-chloro-3-(2-dimethylamino-ethoxy)-phenyl]-benzenesulfonamide;

2,6-Dichloro-N-[4-chloro-3-(2-dimethylamino-ethoxy)-phenyl]-4-trifluoromethyl-benzenesulfonamide;

N-[4-Iodo-3-(2-dimethylamino-ethoxy)-phenyl]-3,4-dimethoxy-benzene sulfonamide;

2-Bromo-N-[4-chloro-3-(2-dimethylamino-ethoxy)-phenyl]-4,5-dimethoxy-benzenesulfonamide;

2,4-Dichloro-N-[4-chloro-3-(2-dimethylamino-ethoxy)-phenyl]-benzenesulfonamide;

2-Methyl-4-bromo-N-[4-chloro-3-(2-dimethylamino-ethoxy)-phenyl]-benzenesulfonamide;

2,6-Dimethyl-4-bromo-N-[4-chloro-3-(2-dimethylamino-ethoxy)-phenyl]-benzenesulfonamide;

3-Methoxy-4-bromo-N-[4-chloro-3-(2-dimethylamino-ethoxy)-phenyl]-benzenesulfonamide;

2,4-Dichloro-5-methyl-N-[4-chloro-3-(2-dimethylamino-ethoxy)-phenyl]-benzenesulfonamide;

3-Nitro-4-chloro-N-[4-chloro-3-(2-dimethylamino-ethoxy)-phenyl]-benzenesulfonamide;

2-Nitro-4-trifluoromethyl-N-[4-chloro-3-(2-dimethylamino-ethoxy)-phenyl]-benzenesulfonamide;

4-Chlorophenyl-N-[4-chloro-3-(2-dimethylamino-ethoxy)-phenyl]-benzenesulfonamide;

5-Chloro-N-[4-chloro-3-(2-dimethylamino-ethoxy)-phenyl]-1-naphthalenesulfonamide;

4-Bromo-5-chloro-N-[4-chloro-3-(2-dimethylamino-ethoxy)-phenyl]-2-thiophenesulfonamide;

3-Bromo-5-chloro-N-[4-chloro-3-(2-dimethylamino-ethoxy)-phenyl]-2-thiophenesulfonamide;

4-Nitro-5-chloro-N-[4-chloro-3-(2-dimethylamino-ethoxy)-phenyl]-2-thiophenesulfonamide;

4,5-Dichloro-N-[4-iodo-3-(2-dimethylamino-ethoxy)-phenyl]-2-thlophenesulfonamide;

7-Chloro-N-[4-iodo-3-(2-dimethylamino-ethoxy)-phenyl]-benzo[1 ,2,5]oxadiazole-4-sulfonamide;

5-Bromo-6-chloro-N-[4-chloro-3-(2-dimethylamino-ethoxy)-phenyl]-3-pyridinesulfonamide;

2,4-Dibromo-5-methoxy-N-[4-chloro-3-(2-dimethylamino-ethoxy)-phenyl]-benzenesulfonamide;

2-Methyl-4,5-dimethoxy-N-[4-chloro-3-(2-dimethylamino-ethoxy)-phenyl]-benzenesulfonamide;

2,6-Dichloro-4-trifluoromethoxy-N-[4-chloro-3-(2-diethylamino-ethoxy)-phenyl]-benzenesulfonamide;

4,5-Dibromo-N-[4-chloro-3-(2-diethylamino-ethoxy)-phenyl]-2-thiophenesulfonamide;

2-Bromo-4,5-dimethoxy-N-[4-chloro-3-(2-diethylamino-ethoxy)-phenyl]-benzenesulfonamide;

3,4-Dimethoxy-N-[4-chloro-3-(3-dimethylamino-propyl)-phenyl]-benzenesulfonamide;

3,4-Dimethoxy-N-[4-chloro-3-(2-diethylamino-ethoxy)-phenyl]-benzenesulfonamide;

2-Chloro-4,5-dimethoxy-N-[4-chloro-3-(2-diethylamino-ethoxy)-phenyl]-benzenesulfonamide; and

2-Chloro-4,5-dimethoxy-N-[4-chloro-3-(2-dimethylamino-ethoxy)-phenyl]-benzenesulfonamide.

Compounds of Formula (I) were prepared as outlined in Scheme 1.

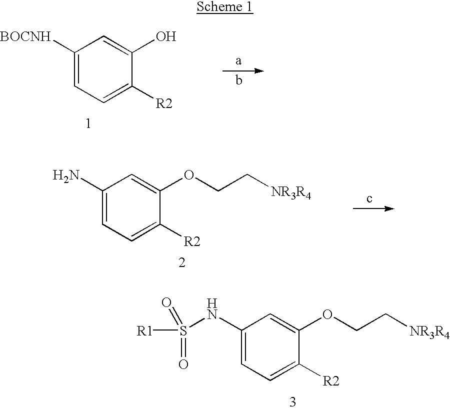

Conditions: a) ClCH₂CH₂NR₃R₄-hydrochloride, potassium carbonate, water/1,2-dimethoxyethane, reflux; b) HCl; c) R₁SO₂Cl, CHCl₃, ambient temperature. (R₁, R₃ and R₄ as defined above)

For example, phenol 1 was alkylated with various dialkylaminoethyl chlorides and the resulting ethers deprotected to provide the anilines 2. Subsequent sulfonylation of the anilines furnished the target compounds 3.

With appropriate manipulation, including the use of alternative nitrogen protecting group(s), the synthesis of the remaining compounds of Formula (I) was accomplished by methods analogous to those above and to those described in the Experimental section.

In order to use a compound of the Formula (I) or a pharmaceutically acceptable salt thereof for the treatment of humans and other mammals it is normally formulated in accordance with standard pharmaceutical practice as a pharmaceutical composition.

Compounds of Formula (I) and their pharmaceutically acceptable salts may be administered in a standard manner for the treatment of the indicated diseases, for example orally, parenterally, sub-lingually, transdermally, rectally, via inhalation or via buccal administration.

Compounds of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, agar, pectin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils and are incorporated in a soft gelatin capsule shell.

Typical parenteral compositions consist of a solution or suspension of the compound or salt in a sterile aqueous or non-aqueous carrier optionally containing a parenterally acceptable oil, for example polyethylene glycol, polyvinylpyrrolidone, lecithin, arachis oil, or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (1) or a pharmaceutically acceptable salt thereof which is active when administered in this way, with a binding and/or lubricating agent, for example polymeric glycols, gelatins, cocoa-butter or other low melting vegetable waxes or fats or their synthetic analogues.

Typical transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer to themselves a single dose.

Each dosage unit for oral administration contains suitably from 0.1 mg to 500 mg/Kg, and preferably from 1 mg to 100 mg/Kg, and each dosage unit for parenteral administration contains suitably from 0.1 mg to 100 mg, of a compound of Formula (I) or a pharmaceutically acceptable salt thereof calculated as the free acid. Each dosage unit for intranasal administration contains suitably 1400 mg and preferably 10 to 200 mg per person. A topical formulation contains suitably 0.01 to 1.0% of a compound of Formula (I).

The daily dosage regimen for oral administration is suitably about 0.01 mg/Kg to 40 mg/Kg, of a compound of Formula (I) or a pharmaceutically acceptable salt thereof calculated as the free acid. The daily dosage regimen for parenteral administration is suitably about 0.001 mg/Kg to 40 mg/Kg, of a compound of the Formula (I) or a pharmaceutically acceptable salt thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

These sulphonamide analogs may be used for the treatment of congestive heart failure, stroke, ischemic heart disease (angina, myocardial ischemia), cardiac arrhythmia, hypertension (essential and pulmonary), COPD, restenosis, asthma, neurogenic inflammation and metabolic vasculopathies, addiction, schizophrenia, impulsivity, anxiety, stress, depression, neuromuscular function, and diabetes.

The urotensin antagonist may be administered alone or in conjunction with one or more other therapeutic agents, said agents being selected from the group consisting of endothelin receptor antagonists, angiotensin converting enzyme (ACE) inhibitors, vasopeptidase inhibitors, diuretics, digoxin, and dual non-selective β-adrenoceptor and α₁-adrenoceptor antagonists.

No unacceptable toxicological effects are expected when compounds of the invention are administered in accordance with the present invention.

The biological activity of the compounds of Formula (I) are demonstrated by the following tests:

Radioligand Binding:

HEK-293 cell membranes containing stable cloned human and rat GPR-14 (20 ug/assay) were incubated with 200 pM [125I] h-U-II (200 Ci/mmol⁻¹ in the presence of increasing concentrations of test compounds in DMSO (0.1 nM to 10 uM), in a final incubation volume of 200 ul (20 mM Tris-HCl, 5 mM MgCl2). Incubation was done for 30 minutes at room temperature followed by filtration GF/B filters with Brandel cell harvester. ¹²⁵I labeled U-U binding was quantitated by gamma counting. Nonspecific binding was defined by ¹²⁵¹I U-II binding in the presence of 100 nM of unlabeled human U-II. Analysis of the data was performed by nonlinear least square fitting.

Ca²⁺-Mobilization:

A microtitre plate based Ca²⁺-mobilization FLIPR assay (Molecular Devices, Sunnyvale, Calif.) was used for the functional identification of the ligand activating HEK-293 cells expressing (stable) recombinant GPR-14. The day following transfection, cells were plated in a poly-D-lysine coated 96 well black/clear plates. After 18-24 hours the media was aspirated and Fluo 3AM-loaded cells were exposed to various concentrations (10 nM to 30 uM) of test compounds followed by h-U-II. After initiation of the assay, fluorescence was read every second for one minute and then every 3 seconds for the following one minute. The inhibitory concentration at 50% (IC50) was calculated for various test compounds.

Inositol Phosphates Assays:

HEK-293-GPR14 cells in T150 flask were prelabeled overnight with 1 uCi myo-[³H] inositol per ml of inositol free Dulbecco's modified Eagel's medium. After labeling, the cells were washed twice with Dulbecco's phosphate-buffered saline (DPBS) and then incubated in DPBS containing 10 mM LiCl for 10 min at 37° C. The experiment was initiated by the addition of increasing concentrations of h-U-II (1 pM to 1 μM ) in the absence and presence of three different concentrations (0.3, 1 and 10 uM) of test compounds and the incubation continued for an additional 5 min at 37° C. after which the reaction was terminated by the addition of 10% (final concentration) trichloroacetic acid and centrifugation. The supernatants were neutralized with 100 ul of 1 M Trizma base and the inositol phosphates were separated on AG 1-X8 columns (0.8 ml packed, 100-200 mesh) in formate phase. Inositol monophosphate was eluted with 8 ml of 200 mM ammonium formate. Combined inositol di and tris phosphate was eluted with 4 ml of 1M ammonium formate/0.1 M formic acid. Eluted fractions were counted in beta scintillation counter. Based on shift from the control curve K_{B} was calculated.

Activity for the compounds of this invention range from (radioligand binding assay): Ki=50 nM−10000 nM (example 8 Ki=1300 nM) The following Examples are illustrative but not limiting embodiments of the present invention.

### EXAMPLE 1

N-[4-Chloro-3-(2-dimethylamino-ethoxy)phenyl]-3,4-dimethoxy-benzenesulfonamide

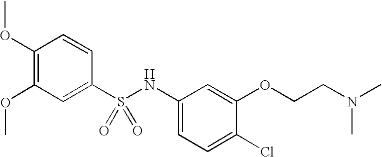

a). 2-Chloro-5-aminophenol

2-Chloro-5-nitroanisole (310 g, 1.7 mol) was taken up in a mixture of 48% HBr (1.5 L) and AcOH (1.2 L) and heated at reflux for 3 days. The dark solution was allowed to cool to room temperature, poured into ice water (10 L), and let stand for 3 h. The resultant dull yellow solid was filtered, washed with water, and dried in vacuo (230 g, 79%): mp 115-117° C.

b). 2-Chloro-5-aminophenol

A solution of 2-chloro-5-nitrophenol (25 g, 0.14 mol) in ethyl acetate (150 mL) was treated with 5% Pt/C (250 mg) and the mixture shaken under a hydrogen atmosphere (30 psi) for 4 h. The mixture was filtered through Celiteg and the residue washed well with hot ethyl acetate. The filtrate was treated with activated charcoal and re-filtered as above. Evaporation of the ethyl acetate gave a solid (19.8 g, 98%).

c). 4-Chloro-3-hydroxyphenylcarbamic acid tert-butyl ester

To a solution of 2-chloro-5-aminophenol (20 g, 0.14 mol) in THF (150 mL) was added a solution of di-tert-butyl dicarbonate (33 g, 0.15 mol) in THF (150 mL). The reaction was heated at reflux for 6 h, at which time it was allowed to cool to room temperature. The solvent was removed in vacuo and the residue diluted with ether (500 mL) and washed with 1 M citric acid (2×300 mL). The aqueous washings were extracted with ether (300 mL) and the combined organics washed with brine (300 mL), dried (MgSO₄). and concentrated. The resultant brown solid was triturated with hexanes and dried in vacuo to give 33 g (97%) of the title compound: mp 103-106° C.

d). 3-[2-(N,N-Dimethylamino)ethoxy]-4-chloroaniline

To a solution of 4-chloro-3-hydroxyphenylcarbamic acid tert-butyl ester (140 mg, 0.57 mmol) in 4:1 DME/water (5 mL) was added dimethylaminoethyl chloride hydrochloride (90 mg, 0.63 mmol) and K₂CO₃ (320 mg, 2.3 mmol). The reaction mixture was heated at reflux for 16 h, at which time it was allowed to cool to room temperature. The DME was removed in vacuo and the residue treated with 6 N HCl (2 mL). The resultant mixture was stirred at room temperature for 2 h, at which time it was diluted with water (5 mL) and washed with EtOAc (5 mL). The aqueous layer was basified with solid K₂CO₃ and extracted with EtOAc (2×10 mL). The EtOAc layers were washed with brine (10 mL), dried (MgSO₄), and concentrated to give 60 mg (50%) of the title compound.

e). N-[4-Chloro-3-(2-dimethylamino-ethoxy)phenyl]-3,4-dimethoxy-benzenesulfonamide

3-[2-(N,N-Dimethylamino)ethoxy]4-chloroaniline (1.00 g, 4.66 mmol) was dissolved in 15 mL CHCl₃. A solution of 3,4-dimethoxybenzenesulfonyl chloride (1.10 g, 4.66 mmol) in 14 mL CHCl₃ was added and the solution was allowed to stir overnight. Diethyl ether was added to the cloudy white mixture and the white product (1.97 g, 94%) was filtered and dried. Recrystallisation from hot methanol gave sparkling white crystals which were filtered and dried: mp 228-229° C.; MS (ES+) m/e 415 [M+H]⁺

The compounds of Examples 2-6 were prepared by using the general procedure(s) of Example I above with appropriate substitution of reactants:

### EXAMPLE 2

4,5-Dibromo-thiophene-2-sulfonic acid [4-chloro-3-(2-dimethylamino-ethoxy)-phenyl]-amide.

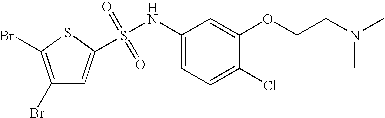

Prepared from 4,5-dibromo-thiophene-2-sulfonyl chloride and 3-[2-(N,N-dimethylamino)ethoxy]-4-chloroaniline. MS (ES+) m/e 517 [M+H]⁺.

### EXAMPLE 3

3,4-Dibromo-N-[4-chloro-3-(2-dimethylamino-ethoxy)-phenyl]-benzenesulfonamide.

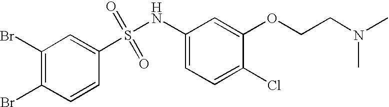

Prepared from 3,4-dibromobenzenesulfonyl chloride and 3-[2-(N,N-dimethylamino)ethoxy]-4-chloroaniline. MS (ES+) m/e 511 [M+H]⁺.

### EXAMPLE 4

2,6-Trichloro-N-[4-chloro-3-(2-dimethylamino-ethoxy)-phenyl]-benzenesulfonamide.

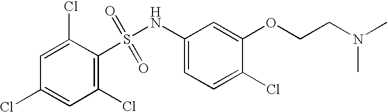

Prepared from 2,4,6-trichlorobenzenesulfonyl chloride and 3-[2-(N,N-dimethylamino)ethoxy]-4-chloroaniline. MS (ES+) m/e 457 [M+H]⁺.

### EXAMPLE 5

2,6-Dichloro-N-[4-chloro-3-(2-dimethylamino-ethoxy)-phenyl]-4-trifluoromethyl-benzenesulfonamide.

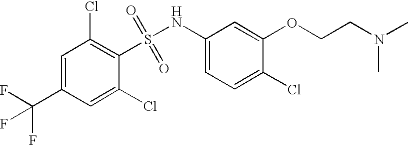

Prepared from 2,6-Dichloro-4-trifluoromethylbenzenesulfonyl chloride and 3-[2-(N,N-dimethylamino)ethoxy]-4-chloroaniline. MS (ES+) n/e 491 [M+H]⁺.

### EXAMPLE 6

N-[3-(2-Dimethylamino-ethoxy)-4-iodo-phenyl]-3,4-dimethoxy-benzenesulfonamide.

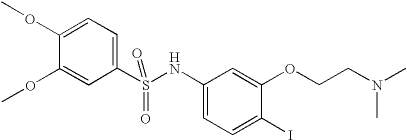

a). N-[3-(2-Dimethylamino-ethoxy)-4-iodo-phenyl]-acetamide

2-Iodo-5-acetamidophenol (2.15 g, 7.76 mmol) was dissolved in 1,2-dimethoxyethane (30 mL). 2-Dimethylaminoethyl chloride hydrochloride (1 eq, 7.76 mmol, 1.12 g) was added, followed by a solution of potassium carbonate (4 eq, 31.0 mmol, 4.30 g) in water (8 mL). The solution was heated to reflux, stirring at this temperature for 22 hours. The 1,2-dimethoxyethane was evaporated in vacuo and the residue was acidified to pH 1 using 3N hydrochloric acid. The mixture was washed 2 x ethyl acetate, and the aqueous portion basified to pH 11 using solid potassium carbonate. It was extracted 2× ethyl acetate, dried over magnesium sulfate, filtered, and concentrated to afford the product (1.53 g, 57%) as a rust-colored oil.

MS (ES+) m/e 349 [M+H]⁺.

b). 3-(2-Dimethylamino-ethoxy)-4-iodo-phenylamine

To a solution of the compound of Example 1(a) (1.52 g, 4.39 mmol) in ethanol (22 mL) was added 10% aqueous sodium hydroxide solution (29 mL). The mixture was heated to reflux and allowed to stir at this temperature for 16 hours. It was cooled to room temperature and concentrated in vacuo. The residue was extracted 2 x ethyl acetate, dried over magnesium sulfate, filtered, and concentrated to furnish the product (1.13 g, 84%) as a rust-colored oil which solidified upon standing.

MS (ES+) m/e 307 [M+H]⁺.

c). N-[3-(2-Dimethylamino-ethoxy)-4-iodo-phenyl]-3,4-dimethoxy-benzenesulfonamide

To a solution of the compound of Example 1(b) (0.25 g, 0.81 mmol) in N,N-dimethylformamide (4 mL) was added 3,4-dimethoxybenzenesulfonyl chloride (1 eq, 0.81 mmol, 0.19 g). The pale orange solution was allowed to stir at room temperature for 23 hours. The crude product was purified via Gilson HPLC purification (10-90% acetonitrile/water over 5 minutes) and lyophilized overnight. The resulting hydochloride salt was azeotroped 1× methanol and 1× methylene chloride to furnish the product (0.16 g, 35%) as a fluffy white solid. MS (ES+) m/e 507 (M+H)⁺.

### EXAMPLE 7

2-Bromo-N-[4-chloro-3-(2-dimethylamino-ethoxy)-phenyl]-4,5-dimethoxy-benzenesulfonamide

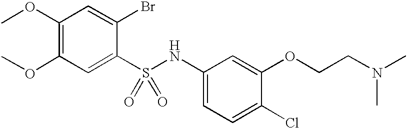

a). 2-Bromo-4,5-dimethoxy-benzenesulfonyl chloride.

To a cooled (0° C.) solution of 4-bromoveratrole (15 mL, 100 mmol) in methylene chloride (100 mL) was added dropwise over 30 minutes chlorosulfonic acid (26 mL, 400 mmol). The resultant solution was allowed to warm to ambient temperature, maintained at this temperature for 3 hours, and then partitioned into a 1:1 methylene chloride/ice water mixture (500 mL). The organic layer was washed with water (2×200 mL) and brine (200 mL), dried (magnesium sulfate), and concentrated to give 2-bromo-4,5-dimethoxybenzenesulfonyl chloride (25 g, 78% yield) as a grey solid.

b). 2-Bromo-N-[4-chloro-3-(2-dimethylamino-ethoxy)-phenyl]-4,5-dimethoxy-benzenesulfonamide.

Prepared from 2-bromo-4,5-dimethoxy-benzenesulfonyl chloride and 3-[2-(N,N-dimethylamino)ethoxy]-4-chloroaniline using the general procedure of Example IE above.

MS (ES+) m/e 494 [M+H]⁺

| | | MS |
|---|---|---|
| | | (ES+) m/e |
| Example | Compound | [M + H] + |
| 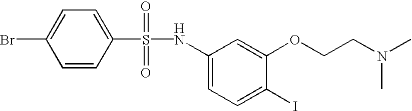 8 | 4-Bromo-N-[4-iodo-3-(2-dimethylamino- ethoxy)-phenyl]-benzenesulfonamide | 525 |
| 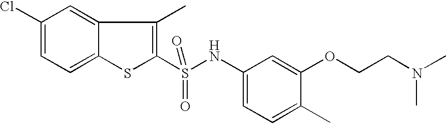 9 | 5-Chloro-3-methyl-N-[4-methyl-3-(2- dimethylamino-ethoxy)-phenyl]-2- benzothiophenesulfonamide | 439 |
| 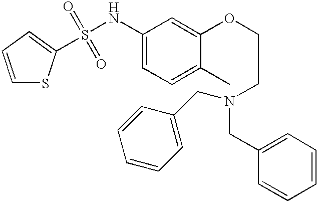 10 | N-[4-Methyl-3-(2-dibenzylamino-ethoxy)- N-phenyl]-2-thiophenesulfonamide | 493 |
| 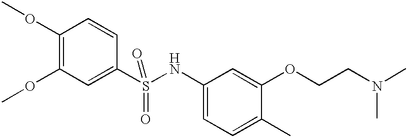 11 | N-[4-Methyl-3-(2-dimethylamino-ethoxy)- phenyl]-3,4-dimethoxy-benzenesulfonamide | 395 |
| 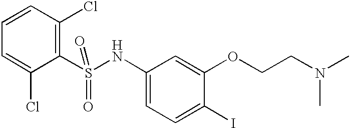 12 | 2,6-Dichloro-N-[4-iodo-3-(2-dimethylamino- ethoxy)-phenyl]-benzenesulfonamide | 515 |
| 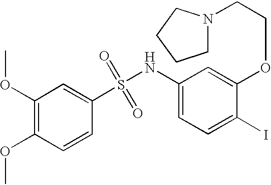 13 | N-[4-Iodo-3-(2-pyrrolidin-1-yl-ethoxy)-phenyl]- 3,4-dimethoxy-benzenesulfonamide | 533 |
| 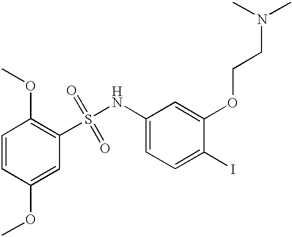 14 | N-[4-Iodo-3-(2-dimethylamino-ethoxy)-phenyl]- 2,5-dimethoxy-benzenesulfonamide | 507 |
| 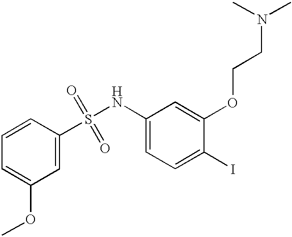 15 | N-[4-Iodo-3-(2-dimethylamino-ethoxy)-phenyl[- 3-methoxy-benzenesulfonamide | 477 |
| 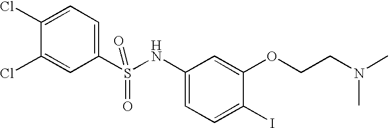 16 | 3,4-Dichloro-N-[4-iodo-3-(2-dimethylamino- ethoxy)-phenyl]-benzenesulfonamide | 515 |
| 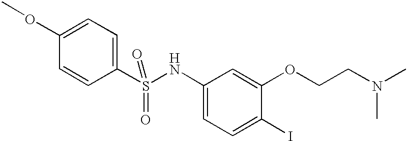 17 | N-[4-Iodo-3-(2-dimethylamino-ethoxy)-phenyl]- 4-methoxy-benzenesulfonamide | 477 |
| 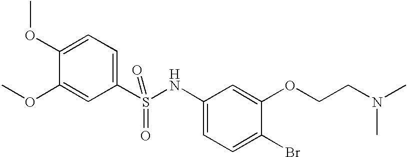 18 | N-[4-Bromo-3-(2-dimethylamino-ethoxy)- phenyl]-3,4-dimethoxy-benzenesulfonamide | 460 |
| 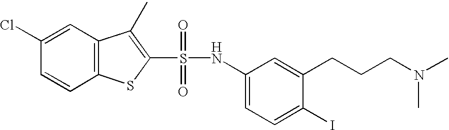 19 | 5-Chloro-3-methyl-N-[4-iodo-3-(3- dimethylamino-propyl)-phenyl]-2- benzothiophenesulfonamide | 549 |
| 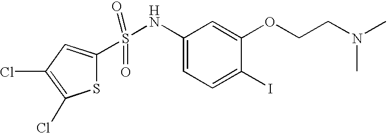 20 | 4,5-Dichloro-N-[4-iodo-3-(2-dimethylamino- ethoxy)-phenyl]-2-thiophenesulfonamide | 521 |
| 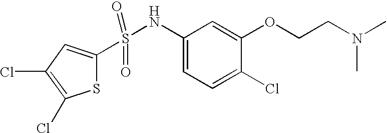 21 | 4,5-Dichloro-N-[4-chloro-3-(2-dimethylamino- ethoxy)-phenyl]-2-thiophenesulfonamide | 429 |
| 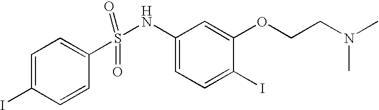 22 | 4-Iodo-N-[4-iodo-3-(2-dimethylamino-ethoxy)- phenyl]-benzenesulfonamide | 573 |
| 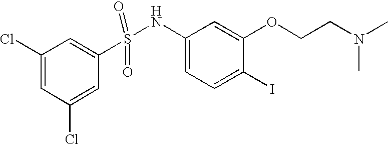 23 | 3,5-Dichloro-N-[4-iodo-3-(2-dimethylamino- ethoxy)-phenyl]-benzenesulfonamide | 515 |
| 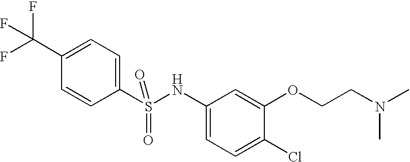 24 | 4-Trifluoromethyl-N-[4-chloro-3-(2- dimethylamino-ethoxy)-phenyl]- benzenesulfonamide | 423 |
| 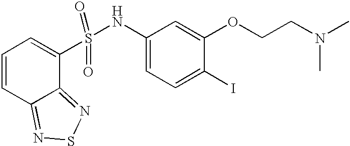 25 | N-[4-iodo-3-(2-dimethylamino-ethoxy)-phenyl]- benzo-2,1,3-thiadiazole-4-sulfonamide | 505 |
| 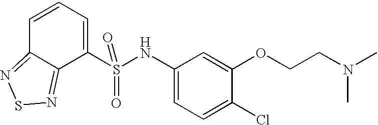 26 | N-[4-chloro-3-(2-dimethylamino-ethoxy)- phenyl]-benzo-2,1,3-thiadiazole-4-sulfonamide | 413 |
| 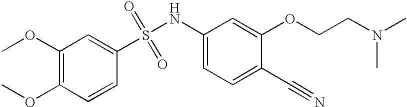 27 | N-[4-Cyano-3-(2-dimethylamino-ethoxy)- phenyl]-3,4-dimethoxy-benzenesulfonamide | 406 |
| 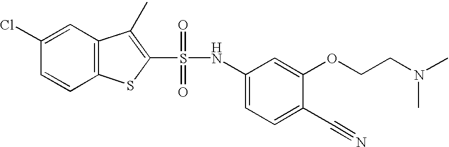 28 | 5-Chloro-3-methyl-N-[4-cyano-3-(2- dimethylamino-ethoxy)-phenyl]-2- benzothiophenesulfonamide | 450 |
| 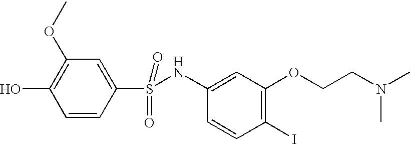 29 | N-[4-Iodo-3-(2-dimethylamino-ethoxy)-phenyl]- 4-hydroxy-3-methoxy-benzenesulfonamide | 493 |
| 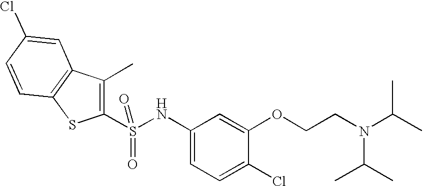 30 | 5-Chloro-3-methyl-N-[4-chloro-3-(2- diisopropylamino-ethoxy)-phenyl]-2- benzothiophenesulfonamide | 515/516 |
| 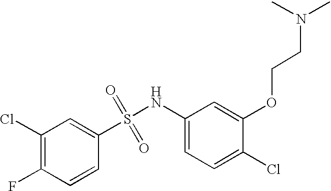 31 | 3-Chloro-N-[4-chloro-3-(2-dimethylamino- ethoxy)-phenyl]-4-fluoro-benzenesulfonamide | 407 |
| 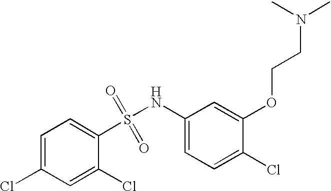 32 | 2,4-Dichloro-N-[4-chloro-3-(2-dimethylamino- ethoxy)-phenyl]-benzenesulfonamide | 423 |
| 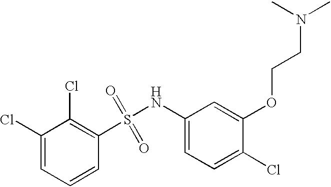 33 | 2,3-Dichloro-N-[4-chloro-3-(2-dimethylamino- ethoxy)-phenyl]-benzenesulfonamide | 423 |
| 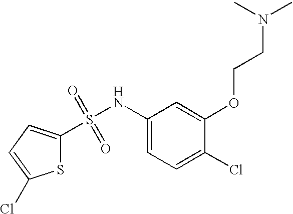 34 | 5-Chloro-N-[4-chloro-3-(2-dimethylamino- ethoxy)-phenyl]-2-thiophenesulfonamide | 395 |
| 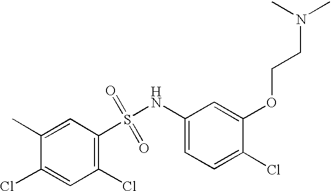 35 | 2,4-Dichloro-N-[4-chloro-3-(2-dimethylamino- ethoxy)-phenyl]-5-methyl-benzenesulfonamide | 437 |
| 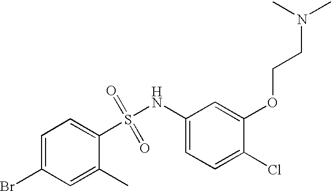 36 | 4-Bromo-N-[4-chloro-3-(2-dimethylamino- ethoxy)-phenyl]-2-methyl-benzenesulfonamide | 447 |
| 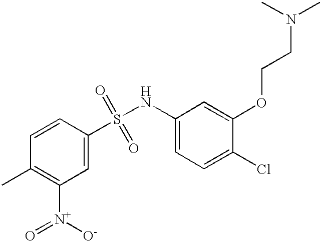 37 | N-[4-Chloro-3-(2-dimethylamino-ethoxy)- phenyl]-4-methyl-3-nitro-benzenesulfonamide | 414 |
| 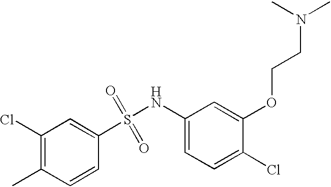 38 | 3-Chloro-N-[4-chloro-3-(2-dimethylamino- ethoxy)-phenyl]-4-methyl-benzenesulfonamide | 403 |
| 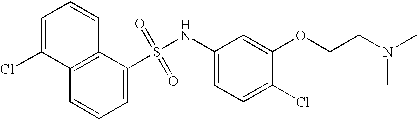 39 | 5-Chloro-N-[4-chloro-3-(2-dimethylamino- ethoxy)-phenyl]-1-naphthalenesulfonamide | 439 |
| 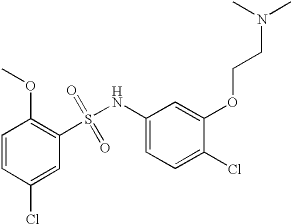 40 | 5-Chloro-N-[4-chloro-3-(2-dimethylamino- ethoxy)-phenyl]-2-methoxy- benzenesulfonamide | 419 |
| 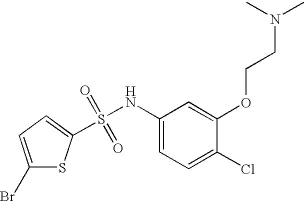 41 | 5-Bromo-N-[4-chloro-3-(2-dimethylamino- ethoxy)-phenyl]-2-thiophenesulfonamide | 439 |
| 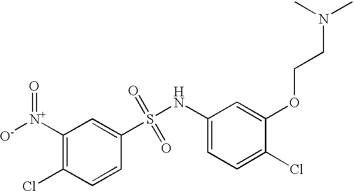 42 | 4-Chloro-N-[4-chloro-3-(2-dimethylamino- ethoxy)-phenyl]-3-nitro-benzenesulfonamide | 434 |
| 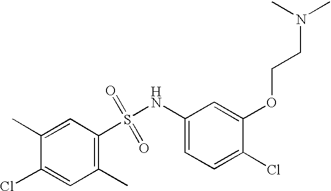 43 | 4-Chloro-N-[4-chloro-3-(2-dimethylamino- ethoxy)-phenyl]-2,5-dimethyl- benzenesulfonamide | 417 |
| 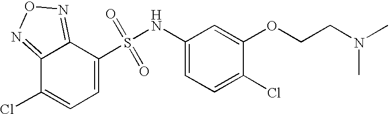 44 | N-[4-chloro-3-(2-dimethylamino-ethoxy)- phenyl]-benzo[1,2,5]oxadiazole-4-sulfonamide | 431 |
| 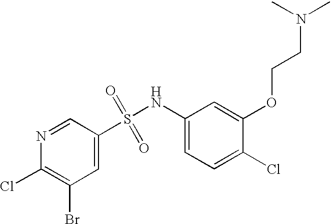 45 | 5-Bromo-6-chloro-N-[4-chloro-3-(2- dimethylamino-ethoxy)-phenyl]-3- pyridinesulfonamide | 468 |
| 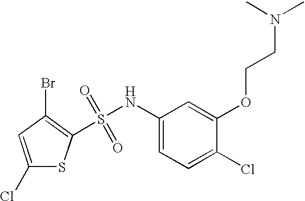 46 | 3-Bromo-5-chloro-N-[4-chloro-3-(2- dimethylamino-ethoxy)-phenyl]-2- thiophenesulfonamide | 473 |
| 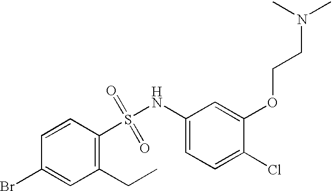 47 | 4-Bromo-N-[4-chloro-3-(2-dimethylamino- ethoxy)-phenyl]-2-ethyl-benzenesulfonamide | 461 |
| 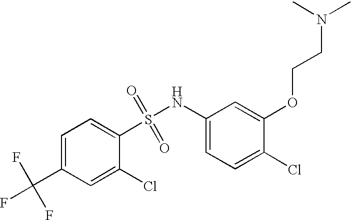 48 | 2-Chloro-N-[4-chloro-3-(2-dimethylamino- ethoxy)-phenyl]-4-trifluoromethyl- benzenesulfonamide | 457 |
| 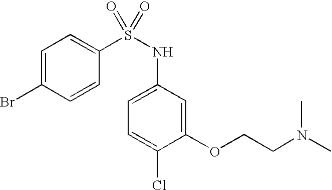 49 | 4-Bromo-N-[4-chloro-3-(2-dimethylamino- ethoxy)-phenyl]-benzenesulfonamide | 433 |
| 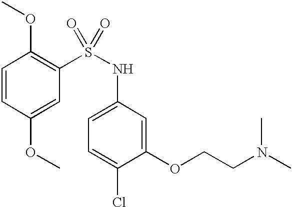 50 | N-[4-Chloro-3-(2-dimethylamino-ethoxy)- phenyl]-2,5-dimethoxy-benzenesulfonamide | 415 |
| 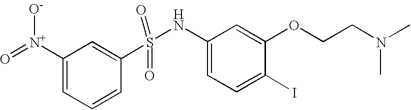 51 | N-[4-Iodo-3-(2-dimethylamino-ethoxy)-phenyl]- 3-nitro-benzenesulfonamide | 491 |
| 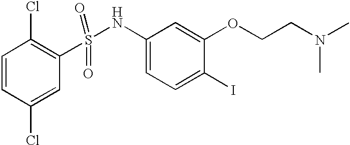 52 | 2,5-Dichloro-N-[4-iodo-3-(2-dimethylamino- ethoxy)-phenyl]-benzenesulfonamide | 514 |
| 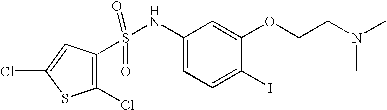 53 | 2,5-Dichloro-N-[4-iodo-3-(2-dimethylamino- ethoxy)-phenyl]-3-thiophenesulfonamide | 520 |
| 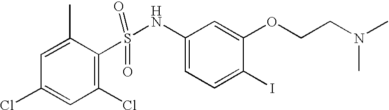 54 | 2,4-Dichloro-N-[4-iodo-3-(2-dimethylamino- ethoxy)-phenyl]-6-methyl-benzenesulfonamide | 528 |
| 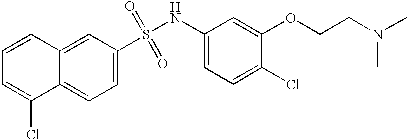 55 | 5-Chloro-N-[4-chloro-3-(2-dimethylamino- ethoxy)-phenyl]-2-naphthalenesulfonamide | 439 |
| 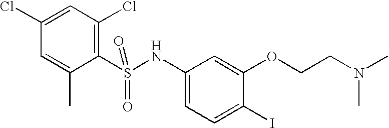 56 | 2,4-Dichloro-6-methyl-N-[4-iodo-3-(2- dimethylamino-ethoxy)-phenyl]- benzenesulfonamide | 529 |
| 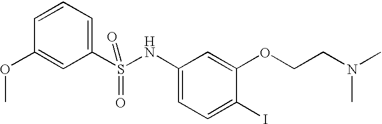 57 | 3-Methoxy-N-[4-iodo-3-(2-dimethylamino- ethoxy)-phenyl]-benzenesulfonamide | 477 |
| 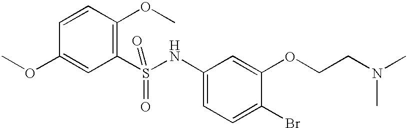 58 | 2,5-Dimethoxy-N-[4-bromo-3-(2- dimethylamino-ethoxy)-phenyl]- benzenesulfonamide | 460 |
| 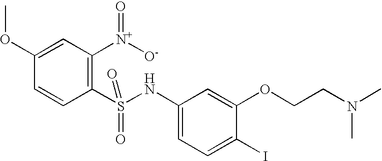 59 | 2-Nitro-4-methoxy-N-[4-iodo-3-(2- dimethylamino-ethoxy)-phenyl]- benzenesulfonamide | 522 |
| 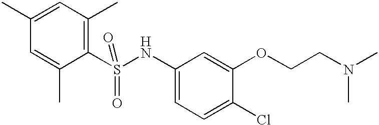 60 | 2,4,6-Trimethyl-N-[4-chloro-3-(2- dimethylamino-ethoxy)-phenyl]- benzenesulfonamide | 397 |
| 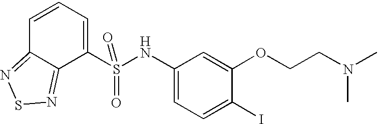 61 | N-[4-Iodo-3-(2-dimethylamino-ethoxy)phenyl]- benzo[1,2,5]-4-thiadiazolesulfonamide | 505 |
| 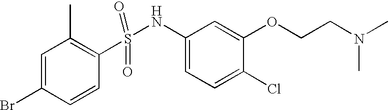 62 | 2-Methyl-4-bromo-N-[4-chloro-3-(2- dimethylamino-ethoxy)-phenyl]- benzenesulfonamide | 447 |
| 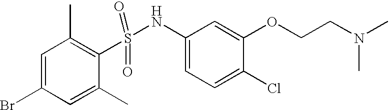 63 | 2,6-Dimethyl-4-bromo-N-[4-chloro-3-(2- dimethylamino-ethoxy)-phenyl]- benzenesulfonamide | 462 |
| 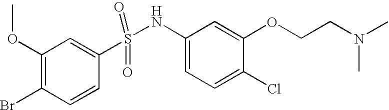 64 | 3-Methoxy-4-bromo-N-[4-chloro-3-(2- dimethylamino-ethoxy)-phenyl]- benzenesulfonamide | 464 |
| 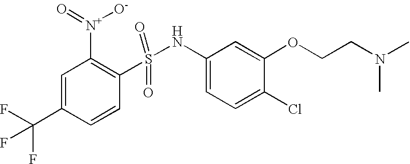 65 | 2-Nitro-4-trifluoromethyl-N-[4-chloro-3-(2- dimethylamino-ethoxy)-phenyl]- benzenesulfonamide | 468 |
| 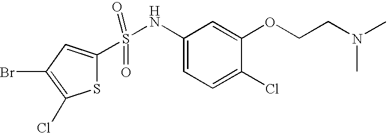 66 | 4-Bromo-5-chloro-N-[4-chloro-3-(2- dimethylamino-ethoxy)-phenyl]-2- thiophenesulfonamide | 474 |
| 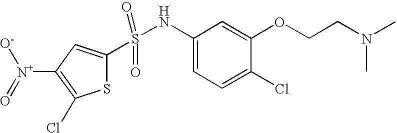 67 | 4-Nitro-5-chloro-N-[4-chloro-3-(2- dimethylamino-ethoxy)-phenyl]-2- thiophenesulfonamide | 440 |
| 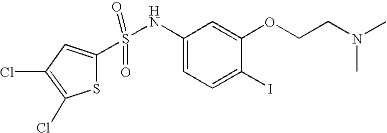 68 | 4,5-Dichloro-N-[4-iodo-3-(2-dimethylamino- ethoxy)-phenyl]-2-thiophenesulfonamide | 521 |
| 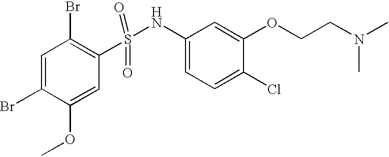 69 | 2,4-Dibromo-5-methoxy-N-[4-chloro-3-(2- dimethylamino-ethoxy)-phenyl]- benzenesulfonamide | 543 |
| 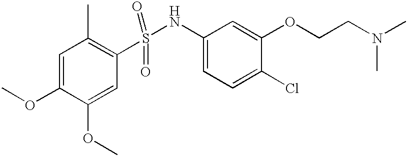 70 | 2-Methyl-4,5-dimethoxy-N-[4-chloro-3-(2- dimethylamino-ethoxy)-phenyl]- benzenesulfonamide | 429 |
| 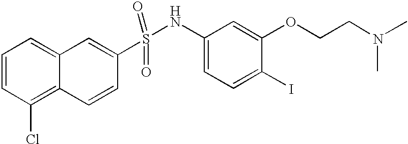 71 | 5-Chloro-N-[4-iodo-3-(2-dimethylamino- ethoxy)-phenyl]-2-naphthalenesulfonamide | 531 |
| 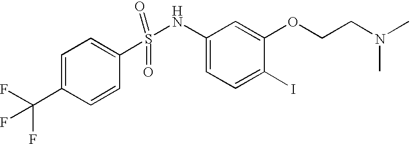 72 | 4-Trifluoromethyl-N-[4-iodo-3-(2- dimethylamino-ethoxy)-phenyl]- benzenesulfonamide | 515 |
| 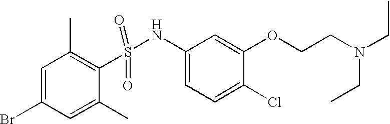 73 | 2,6-Dimethyl-4-bromo-N-[4-chloro-3-(2- diethylamino-ethoxy)-phenyl]- benzenesulfonamide | 490 |
| 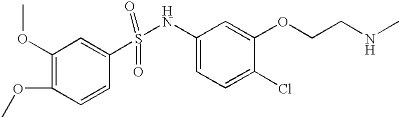 74 | 3,4-Dimethoxy-N-[4-chloro-3-(2-methylamino- ethoxy)-phenyl]-benzenesulfonamide | 401 |
| 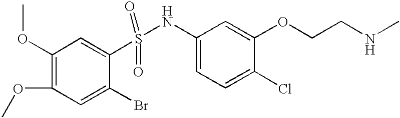 75 | 2-Bromo-4,5-dimethoxy-N-[4-chloro-3-(2- methylamino-ethoxy)-phenyl]- benzenesulfonamide | 480 |
| 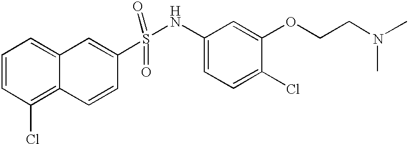 76 | 5-Chloro-N-[4-chloro-3-(2-dimethylamino- ethoxy)-phenyl]-2-naphthalenesulfonamide | 439 |
| 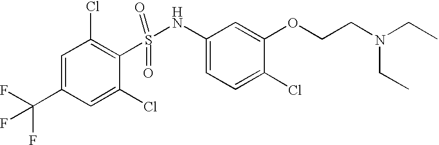 77 | 2,6-Dichloro-4-trifluoromethoxy-N-[4-chloro-3- (2-diethylamino-ethoxy)-phenyl]- benzenesulfonamide | 519 |
| 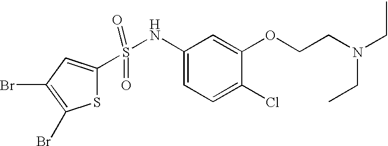 78 | 4,5-Dibromo-N-[4-chloro-3-(2-diethylamino- ethoxy)-phenyl]-2-thiophenesulfonamide | 547 |
| 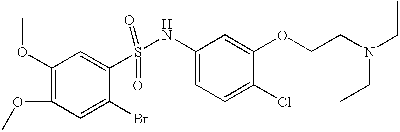 79 | 2-Bromo-4,5-dimethoxy-N-[4-chloro-3-(2- diethylamino-ethoxy)-phenyl]- benzenesulfonamide | 522 |
| 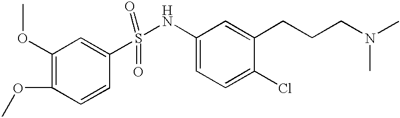 80 | 3,4-Dimethoxy-N-[4-chloro-3-(3- dimethylamino-propyl)-phenyl]- benzenesulfonamide | 413 |
| 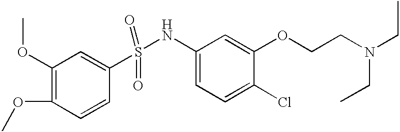 81 | 3,4-Dimethoxy-N-[4-chloro-3-(2-diethylamino- ethoxy)-phenyl]-benzenesulfonamide | 443 |
| 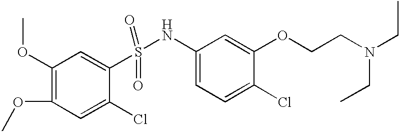 82 | 2-Chloro-4,5-dimethoxy-N-[4-chloro-3-(2- diethylamino-ethoxy)-phenyl]- benzenesulfonamide | 477 |
| 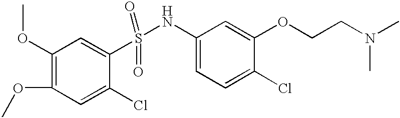 83 | 2-Chloro-4,5-dimethoxy-N-[4-chloro-3-(2- dimethylamino-ethoxy)-phenyl]- benzenesulfonamide | 449 |
| 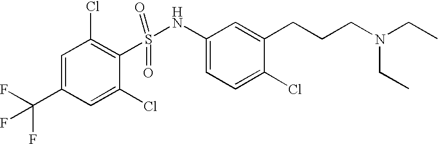 84 | 2,6-Dichloro-N-[4-chloro-3-(3-diethylamino- propyl)-phenyl]-4- trifluoromethylbenzenesulfonamide | 516 |
| 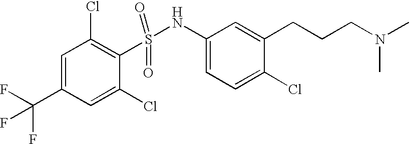 85 | 2,6-Dichloro-N-[4-chloro-3-(3-dimethylamino- propyl)-phenyl]-4- trifluoromethylbenzenesulfonamide | 488 |
| 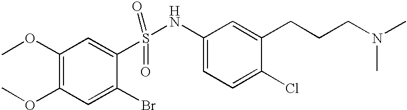 86 | 4,5-Dimethoxy-N-[4-chloro-3-(3- dimethylamino-propyl)-phenyl]-2- bromobenzenesulfonamide | 491 |
| 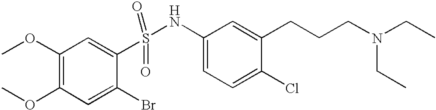 87 | 4.5-Dimethoxy-N-[4-chloro-3-(3-diethylamino- propyl)-phenyl]-2-bromobenzenesulfonamide | 519 |
| 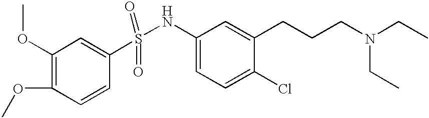 88 | 3,4-Dimethoxy-N-[4-chloro-3-(3-diethylamino- propyl)-phenyl]benzenesulfonamide | 440 |
| 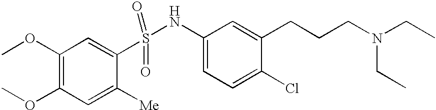 89 | 4,5-Dimethoxy-N-[4-chloro-3-(3-diethylamino- propyl)-phenyl]-2-methylbenzenesulfonamide | 454 |
| 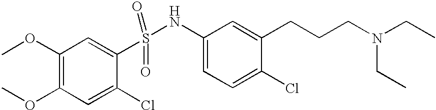 90 | 4,5-Dimethoxy-N-[4-chloro-3-(3-diethylamino- propyl)-phenyl]-2-chlorobenzenesulfonamide | 474 |
| 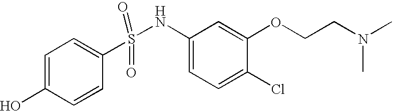 91 | N-[4-Chloro-3-(2-dimethylamino-ethoxy)- phenyl]-4-hydroxybenzenesulfonamide | 371 |
| 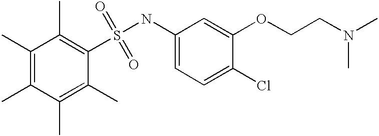 92 | N-[4-Chloro-3-(2-dimethylamino-ethoxy)- phenyl]-2,3,4,5,6-pentamethyl- benzenesulfonamide | 425 |
| 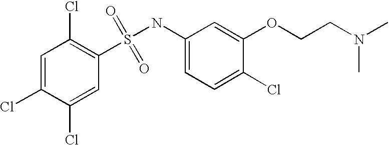 93 | 2,4,5-Trichloro-N-[4-chloro-3-(2- dimethylamino-ethoxy)-phenyl]- benzenesulfonamide | 457 |
| 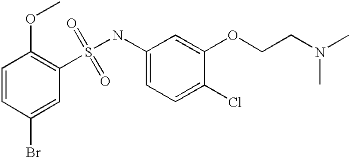 94 | 5-Bromo-N-[4-chloro-3-(2-dimethylamino- ethoxy)-phenyl]-2-methoxy- benzenesulfonamide | 463 |
| 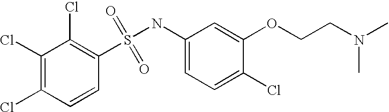 95 | 2,3,4-Trichloro-N-[4-chloro-3-(2- dimethylamino-ethoxy)-phenyl]- benzenesulfonamide | 457 |
| 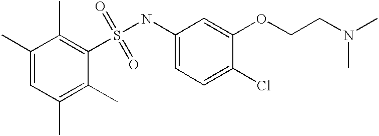 96 | N-[4-Chloro-3-(2-dimethylamino-ethoxy)- phenyl]-2,3,5,6-tetramethyl- benzenesulfonamide | 411 |
| 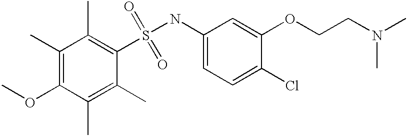 97 | N-[4-Chloro-3-(2-dimethylamino-ethoxy)- phenyl]-4-methoxy-2,3,6-trimethyl- benzenesulfonamide | 427 |
| 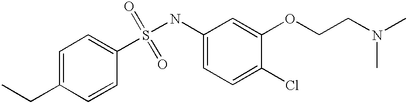 98 | N-[4-Chloro-3-(2-dimethylamino-ethoxy)- phenyl]-4-ethyl-benzenesulfonamide | 383 |
| 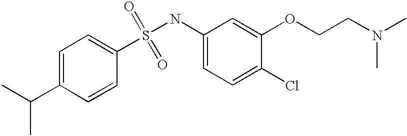 99 | N-[4-Chloro-3-(2-dimethylamino-ethoxy)- phenyl]-4-isopropyl-benzenesulfonamide | 397 |
| 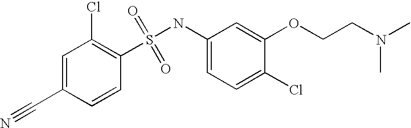 100 | 2-Chloro-N-[4-chloro-3-(2-dimethylamino- ethoxy)-phenyl]4-cyano-benzenesulfonamide | 414 |
| 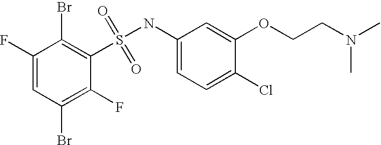 101 | 2,5-Dibromo-N-[4-chloro-3-(2-dimethylamino- ethoxy)-phenyl]-3,6-difluoro- benzenesulfonamide | 547 |
| 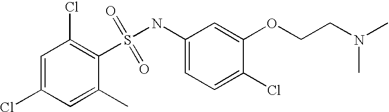 102 | 2,4-Dichloro-N-[4-chloro-3-(2-dimethylamino- ethoxy)-phenyl]-6-methyl-benzenesulfonamide | 437 |
| 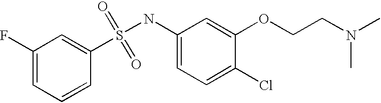 103 | N-[4-Chloro-3-(2-dimethylamino-ethoxy)- phenyl]-3-fluoro-benzenesulfonamide | 373 |
| 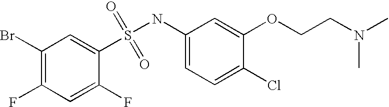 104 | 5-Bromo-N-[4-chloro-3-(2-dimethylamino- ethoxy)-phenyl]-2,4-difluoro- benzenesulfonamide | 469 |
| 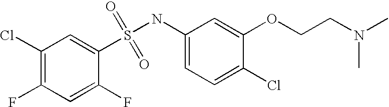 105 | 5-Chloro-N-[4-chloro-3-(2-dimethylamino- ethoxy)-phenyl]-2,4-difluoro- benzenesulfonamide | 425 |
| 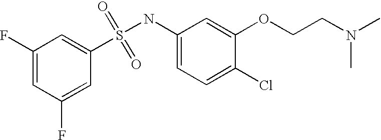 106 | N-[4-Chloro-3-(2-dimethylamino-ethoxy)- phenyl]-3,5-difluoro-benzenesulfonamide | 391 |
| 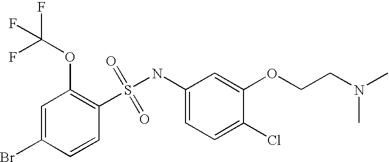 107 | 4-Bromo-N-[chloro-(2-dimethylamino-ethoxy)- phenyl]-2-trifluoromethoxy- benzenesulfonamide | 517 |
| 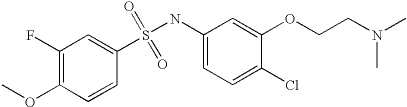 108 | N-[4-Chloro-3-(2-dimethylamino-ethoxy)- phenyl]-3-fluoro-4-methoxy- benzenesulfonamide | 403 |
| 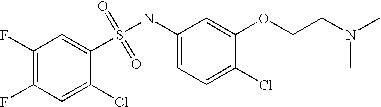 109 | 2-Chloro-N-[4-chloro-3-(2-dimethylamino- ethoxy)-phenyl]-4,5-difluoro- benzenesulfonamide | 425 |
| 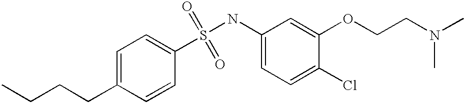 110 | 4-Butyl-N-[4-chloro-3-(2-dimethylamino- ethoxy)-phenyl]-benzenesulfonamide | 411 |
| 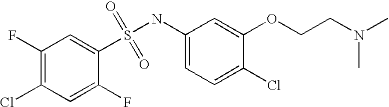 111 | 4-Chloro-N-[4-chloro-3-(2-dimethylamino- ethoxy)-phenyl]-2,5-difluoro- benzenesulfonamide | 425 |
| 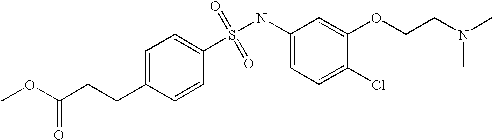 112 | 3-{4-[4-Chloro-3-(2-dimethylamino-ethoxy)- phenylsulfamoyl]-phenyl}-propionic acid methyl ester | 441 |
| 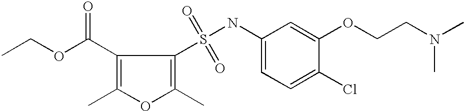 113 | 4-[4-Chloro-3-(2-dimethylamino-ethoxy)- phenylsulfamoyl]-2,5-dimethyl-furan-3- carboxylic acid ethyl ester | 445 |
| 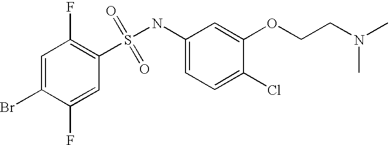 114 | 4-Bromo-N-[4-chloro-3-(2-dimethylamino- ethoxy)-phenyl]-2,5-difluoro- benzenesulfonamide | 469 |
| 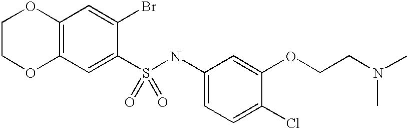 115 | 7-Bromo-2,3-dihydro-benzo[1,4]dioxine-6- sulfonic acid [4-chloro-3-(2-dimethylamino- ethoxy)-phenyl]-amide | 491 |
| 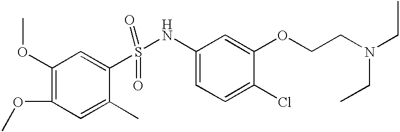 116 | N-[4-chloro-3-(2-diethylamino-ethoxy)- phenyl]-4,5-dimethoxy-2-methyl- benzenesulfonamide | 457 |
| 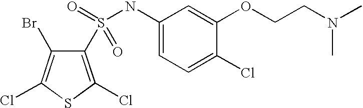 117 | 4-Bromo-2,5-dichloro-thiophene-3-sulfonic acid [4-chloro-3-(2-dimethylamino-ethoxy)-phenyl]- amide | 507 |
| 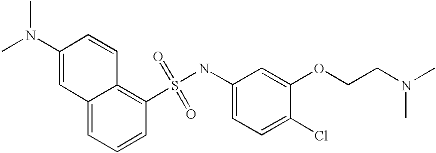 118 | 3-Dimethylamino-naphthalene-1-sulfonic acid [4-chloro-3-(2-dimethylamino-ethoxy)-phenyl]- amide | 448 |

### EXAMPLE 119

Formulations for pharmaceutical use incorporating compounds of the present invention can be prepared in various forms and with numerous excipients. Examples of such formulations are given below.

| Tablets/Ingredients | | Per Tablet | |
|---|---|---|---|
| 1. | Active ingredient | 40 | mg |
| | (Cpd of Form. I) | | |
| 2. | Corn Starch | 20 | mg |
| 3. | Alginic acid | 20 | mg |
| 4. | Sodium Alginate | 20 | mg |
| 5. | Mg stearate | 1.3 | mg |
| | | 2.3 | mg |

Procedure for Tablets:

Step 1: Blend ingredients No. 1, No. 2, No. 3 and No. 4 in a suitable mixer/blender.

Step 2: Add sufficient water portion-wise to the blend from Step I with careful mixing after each addition. Such additions of water and mixing until the mass is of a consistency to permit its conversion to wet granules.

Step 3: The wet mass is converted to granules by passing it through an oscillating granulator using a No. 8 mesh (2.38 mm) screen.

Step 4: The wet granules are then dried in an oven at 140° F. (60° C.) until dry.

Step 5: The dry granules are lubricated with ingredient No. 5.

Step 6: The lubricated granules are compressed on a suitable tablet press.

Inhalant Formulation

A compound of Formula I, (1 mg to 100 mg) is aerosolized from a metered dose inhaler to deliver the desired amount of drug per use.

Parenteral Formulation

A pharmaceutical composition for parenteral administration is prepared by dissolving an appropriate amount of a compound of formula I in polyethylene glycol with heating. This solution is then diluted with water for injections Ph Eur. (to 100 ml). The solution is then sterilized by filtration through a 0.22 micron membrane filter and sealed in sterile containers.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

What is claimed is:

1. 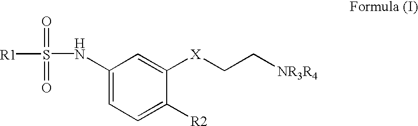
wherein:
R₁ is phenyl, benzothiophenyl, thienyl, furyl, pyrrolyl, pyridinyl, benzthiadiazoyl, benzoxadiazoyl, quinolinyl, or naphthyl, all of which may be substituted or unsubstituted by one, two, three, four or five of the following: halogen, methoxy, OH, NO₂, YCF₃, C₁₋₄ alkyl, C₍₀₋₄₎alkylCO₂C₍₀₋₄₎alkyl, cyano, cycloC₍₁₋₄₎alkylenedioxy, or dimethylamino;
R₂ is halogen, CN or methyl;
R₃ and R₄ are independently hydrogen, C₁₆ alkyl or benzyl; or with the nitrogen form a pyrrolidine or piperidine ring;
X is 0 or CH₂;
Y is a bond or O;
provided the compound of Formula (I) is not 5-Chloro-3-methyl-benzo[b]thiophene-2-sulfonic acid [3-(2-dimethylamino-ethoxy)-4-iodo-phenyl]-amide; or a pharmaceutically acceptable salt thereof.

2. A compound according to claim 1 wherein RI is phenyl, thienyl, pyridinyl, benzthiadiazoyl, benzoxadiazoyl, or naphthyl, all of which may be substituted or unsubstituted by one, two, or three of the following: halogen, methoxy, NO₂, YCF₃, or C₁₋₄ alkyl; R₂ is halogen; R₃ is alkyl; R₄ is alkyl; X is O, and Y is a bond.

3. A compound according to claim 1 wherein R₁ is R₁ is phenyl, thienyl, pyridinyl, benzthiadiazoyl, benzoxadiazoyl, or naphthyl, all of which may be substituted or unsubstituted by one, two, or three of the following: halogen, methoxy, NO₂, YCF₃, or C₁₋₄ alkyl; R₂ is halogen; R₃ is methyl or ethyl; R₄ is methyl or ethyl; X is O, and Y is a bond.

4. A compound according to claim 1 chosen from the group consisting of:
N-[4-Iodo-3-(2-dimethylamino-ethoxy)-phenyl]-3 ,4-dimethoxy-benzenesulfonamide;
4-Bromo-N-[4-iodo-3-(2-dimethylamino-ethoxy)-phenyl]-benzenesulfonamide;
N-[4-Methyl-3-(2-dimethylamino-ethoxy)-phenyl]-3,4-dimethoxy-benzenesulfonamide;
N-[4-Iodo-3-(2-dimethylamino-ethoxy)-phenyl]-3-methoxy-benzenesulfonamide;
N-[4-Bromo-3-(2-dimethylamino-ethoxy)-phenyl]-3,4-dimethoxy-benzenesulfonamide;
N-[4-Chloro-3-(2-dimethylamino-ethoxy)-phenyl]-3 ,4-dimethoxy-benzenesulfonamide;
4,5-Dibromo-N-[4-chloro-3-(2-dimethylamino-ethoxy)-phenyl]-2-thiophenesulfonamide;
3,4-Dibromo-N-[4-chloro-3-(2-dimethylamino-ethoxy)-phenyl]-benzenesulfonamide;
2,4,6-Trichloro-N-[4-chloro-3-(2-dimethylamino-ethoxy)-phenyl]-benzenesulfonamide;
2,6-Dichloro-N-[4-chloro-3-(2-dimethylamino-ethoxy)-phenyl]-4-trifluoromethyl-benzenesulfonamide;
2-Bromo-N-[4-chloro-3-(2-dimethylamino-ethoxy)-phenyl]-4,5-dimethoxy-benzenesulfonamide;
4-Bromo-N-[4-chloro-3-(2-dimethylamino-ethoxy)-phenyl]-benzenesulfonamide;
4-Iodo-N-[4-Iodo-3-(2-dimethylamino-ethoxy)-phenyl]-benzenesulfonamide;
3,5-Dichloro-N-[4-iodo-3-(2-dimethylamino-ethoxy)-phenyl]-benzenesulfonamide;
2,3-Dichloro-N-[4-chloro-3-(2-dimethylamino-ethoxy)-phenyl]-benzenesulfonamide;
3-Chloro-4-fluoro-N-[4-chloro-3-(2-dimethylamino-ethoxy)-phenyl]-benzenesulfonamide;
3-Chloro-4-methyl-N-[4-chloro-3-(2-dimethylamino-ethoxy)-phenyl]-benzenesulfonamide;
2,5-Dimethyl-4-chloro-N-[4-chloro-3-(2-dimethylamino-ethoxy)-phenyl]-benzenesulfonamide;
2-Chloro-4-trifluoromethyl-N-[4-chloro-3-(2-dimethylamino-ethoxy)-phenyl]-benzenesulfonamide;
2,4-Dichloro-6-methyl-N-[4-iodo-3-(2-dimethylamino-ethoxy)-phenyl]-benzenesulfonamide;
3-Methoxy-N-[4-iodo-3-(2-dimethylamino-ethoxy)-phenyl]-benzenesulfonamide;
2,5-Dimethoxy-N-[4-chloro-3-(2-dimethylamino-ethoxy)-phenyl]-benzenesulfonamide;
2,5-Dimethoxy-N-[4-bromo-3-(2-dimethylamino-ethoxy)-phenyl]-benzenesulfonamide;
3-Nitro-N-[4-iodo-3-(2-dimethylamino-ethoxy)-phenyl]-benzenesulfonamide;
2-Nitro-4-methoxy-N-[4-iodo-3-(2-dimethylamino-ethoxy)-phenyl]-benzenesulfonamide;
3-Nitro-4-methyl-N-[4-chloro-3-(2-dimethylamino-ethoxy)-phenyl]-benzenesulfonamide;
2-Ethyl-4-bromo-N-[4-chloro-3-(2-dimethylamino-ethoxy)-phenyl]-benzenesulfonamide;
3,4-Dichlorophenyl-N-[4-chloro-3-(2-dimethylamino-ethoxy)-phenyl]-benzenesulfonamide;
2,4,6-Trimethyl-N-[4-chloro-3-(2-dimethylamino-ethoxy)-phenyl]-benzenesulfonamide;
4-Chloro-N-[4-chloro-3-(2-dimethylamino-ethoxy)-phenyl]-2-naphthalenesulfonamide;
5-Chloro-N-[4-chloro-3-(2-dimethylamino-ethoxy)-phenyl]-2-thiophenesulfonamide;
2,5-Dichloro-N-[4-iodo-3-(2-dimethylamino-ethoxy)-phenyl]-3-thiophenesulfonamide;
5-Bromo-N-[4-chloro-3-(2-dimethylamino-ethoxy)-phenyl]-2-thiophenesulfonamide;
4,5-Dichloro-N-[4-chloro-3-(2-dimethylamino-ethoxy)-phenyl]-2-thiophenesulfonamide;
5-({[[-(4-Chloro-phenyl)-methanoyl]-amino}methyl)-N-[4-chloro-3-(2-dimethylamino-ethoxy)-phenyl]-2-thiophenesulfonamide;
N-[4-Iodo-3-(2-dimethylamino-ethoxy)-phenyl]-benzo[1,2,5]-4-thiadiazolesulfonamide;
2,4-Dichloro-N-[4-chloro-3-(2-dimethylamino-ethoxy)-phenyl]-benzenesulfonamide;
2-Methyl-4-bromo-N-[4-chloro-3-(2-dimethylamino-ethoxy)-phenyl]-benzenesulfonamide;
2,6-Dimethyl-4-bromo-N-[4-chloro-3-(2-dimethylamino-ethoxy)-phenyl]-benzenesulfonamide;
3-Methoxy-4-bromo-N-[4-chloro-3-(2-dimethylamino-ethoxy)-phenyl]-benzenesulfonamide;
2,4-Dichloro-5-methyl-N-[4-chloro-3-(2-dimethylamino-ethoxy)-phenyl]-benzenesulfonamide;
3-Nitro-4-chloro-N-[4-chloro-3-(2-dimethylamino-ethoxy)-phenyl]-benzenesulfonamide;
2-Nitro-4-trifluoromethyl-N-[4-chloro-3-(2-dimethylamino-ethoxy)-phenyl]-benzenesulfonamide;
5-Chloro-N-[4-chloro-3-(2-dimethylamino-ethoxy)-phenyl]-1-naphthalenesulfonamide;
4-Bromo-5-chloro-N-[4-chloro-3-(2-dimethylamino-ethoxy)-phenyl]-2-thiophenesulfonamide;
3-Bromo-5-chloro-N-[4-chloro-3-(2-dimethylamino-ethoxy)-phenyl]-2-thiophenesulfonamide;
4-Nitro-5-chloro-N-[4-chloro-3-(2-dimethylamino-ethoxy)-phenyl]-2-thiophenesulfonamide;
4,5-Dichloro-N-[4-iodo-3-(2-dimethylamino-ethoxy)-phenyl]-2-thiophenesulfonamide;
7-Chloro-N-[4-iodo-3-(2-dimethylamino-ethoxy)-phenyl]-benzo[1,2,5]oxadiazole-4-sulfonamide;
5-Bromo-6-chloro-N-[4-chloro-3-(2-dimethylamino-ethoxy)-phenyl]-3-pyridinesulfonamide;
2,4-Dibromo-5-methoxy-N-[4-chloro-3-(2-dimethylamino-ethoxy)-phenyl]-benzenesulfonamide;
2-Methyl-4,5-dimethoxy-N-[4-chloro-3-(2-dimethylamino-ethoxy)-phenyl]-35 benzenesulfonamide;
2,6-Dimethyl-4-bromo-N-[4-chloro-3-(2-diethylamino-ethoxy)-phenyl]-benzenesulfonamnide;
3,4-Dimethoxy-N-[4-chloro-3-(2-methylamino-ethoxy)-phenyl]-benzenesulfonamide;
2-Bromo-4,5-dimethoxy-N-[4-chloro-3-(2-methylamino-ethoxy)-phenyl]-benzenesulfonamide;
5-Chloro-N-[4-chloro-3-(2-dimethylamino-ethoxy)-phenyl]-2-naphthalenesulfonamide;
2,6-Dichloro-4-trifluoromethoxy-N-[4-chloro-3-(2-diethylamino-ethoxy)-phenyl]-benzenesulfonamide;
4,5-Dibromo-N-[4-chloro-3-(2-diethylamino-ethoxy)-phenyl]-2-thiophenesulfonamide;
2-Bromo-4,5-dimethoxy-N-[4-chloro-3-(2-diethylamino-ethoxy)-phenyl]-benzenesulfonamide;
3,4-Dimethoxy-N-[4-chloro-3-(3-dimethylamino-propyl)-phenyl]-benzenesulfonamide;
3,4-Dimethoxy-N-[4-chloro-3-(2-diethylamino-ethoxy)-phenyl]-benzenesulfonamide;
2-Chloro-4,5-dimethoxy-N-[4-chloro-3-(2-diethylamino-ethoxy)-phenyl]-benzenesulfonamide; and
2-Chloro-4,5-dimethoxy-N-[4-chloro-3-(2-dimethylamino-ethoxy)-phenyl]-benzenesulfonamide.

5. A compund of claim 1 chosen from the group consisting of:
N-[4-Chloro-3-(2-dimethylamino-ethoxy)-phenyl]-3,4-dimethoxy-benzenesulfonamide;
4,5-Dibromo-N-[4-chloro-3-(2-dimethylamino-ethoxy)-phenyl]-2-thiophenesulfonamide;
3,4-Dibromo-N-[4-chloro-3-(2-dimethylamino-ethoxy)-phenyl]-benzenesulfonamide;
2,4,6-Trichloro-N-[4-chloro-3-(2-dimethylamino-ethoxy)-phenyl]-benzenesulfonamide;
2,6-Dichloro-N-[4-chloro-3-(2-dimethylamino-ethoxy)-phenyl]-4-trifluoromethyl-benzenesulfonamide;
N-[4-Iodo-3-(2-dimethylamino-ethoxy)-phenyl]-3,4-dimethoxy-benzenesulfonamide;
2-Bromo-N-[4-chloro-3-(2-dimethylamino-ethoxy)-phenyl]-4,5-dimethoxy-benzenesulfonamide;
2,4-Dichloro-N-[4-chloro-3-(2-dimethylamino-ethoxy)-phenyl]-benzenesulfonamide;
2-Methyl-4-bromo-N-[4-chloro-3-(2-dimethylamino-ethoxy)-phenyl]-benzenesulfonamide;
2,6-Dimethyl-4-bromo-N-[4-chloro-3-(2-dimethylamino-ethoxy)-phenyl]-benzenesulfonamide;
3-Methoxy-4-bromo-N-[4-chloro-3-(2-dimethylamino-ethoxy)-phenyl]-benzenesulfonamide;
2,4-Dichloro-5-methyl-N-[4-chloro-3-(2-dimethylamino-ethoxy)-phenyl]-benzenesulfonamide;
3-Nitro-4-chloro-N-[4-chloro-3-(2-dimethylamino-ethoxy)-phenyl]-benzenesulfonamide;
2-Nitro-4-trifluoromethyl-N-[4-chloro-3-(2-dimethylamino-ethoxy)-phenyl]-benzenesulfonamide;
4-Chlorophenyl-N-[4-chloro-3-(2-dimethylamino-ethoxy)-phenyl]-benzenesulfonamide;
5-Chloro-N-[4-chloro-3-(2-dimethylamino-ethoxy)-phenyl]-1-naphthalenesulfonamide;
4-Bromo-5-chloro-N-[4-chloro-3-(2-dimethylamino-ethoxy)-phenyl]-2-thiophenesulfonamide;
3-Bromo-5-chloro-N-[4-chloro-3-(2-dimethylamino-ethoxy)-phenyl]-2-thiophenesulfonamide;
4-Nitro-5-chloro-N-[4-chloro-3-(2-dimethylamino-ethoxy)-phenyl]-2-thiophenesulfonamide;
4,5-Dichloro-N-[4-iodo-3-(2-dimethylamino-ethoxy)-phenyl]-2-thiophenesulfonamide;
7-Chloro-N-[4-iodo-3-(2-dimethylamino-ethoxy)-phenyl]-benzo[1,2,5]oxadiazole-4-sulfonamide;
5-Bromo-6-chloro-N-[4-chloro-3-(2-dimethylamino-ethoxy)-pbenyl]-3-pyridinesulfonamide;
2,4-Dibromo-5-methoxy-N-[4-chloro-3-(2-dimethylamino-ethoxy)-phenyl]-benzenesulfonamide;
2-Methyl-4,5-dimethoxy-N-[4-chloro-3-(2-dimethylamino-ethoxy)-phenyl]-benzenesulfonamide;
2,6-Dichloro-4-trifluoromethoxy-N-[4-chloro-3-(2-diethylamino-ethoxy)-phenyl]-benzenesulfonamide;
4,5-Dibromo-N-[4-chloro-3-(2-diethylamino-ethoxy)-phenyl]-2-thiophenesulfonamide;
2-Bromo-4,5-dimethoxy-N-[4-chloro-3-(2-diethylamino-ethoxy)-phenyl]-benzenesulfonamide;
3,4-Dimethoxy-N-[4-chloro-3-(3-dimethylamino-propyl)-phenyl]-benzenesulfonamide;
3,4-Dimethoxy-N-[4-chloro-3-(2-diethylamino-ethoxy)-phenyl]-benzenesulfonamide;
2-Chloro-4,5-dimethoxy-N-[4-chloro-3-(2-diethylamino-ethoxy)-phenyl]-benzenesulfonamide; and
2-Chloro-4,5-dimethoxy-N-[4-chloro-3-(2-dimethylamino-ethoxy)-phenyl]-benzenesulfonamide.

6. A pharmaceutical composition comprising a compound of claim 1 and a pharmaceutically acceptable carrier.

7. A method of treating conditions associated with Urotensin-II imbalance by antagonizing the Urotensin-IT receptor which comprises administering to a patient in need thereof, a compound of Formula I of claim 1.

8. A method according to claim 7 wherein the disease is congestive heart failure, stroke, ischemic heart disease, angina, myocardial ischemia, cardiac arrythmias, essential hypertension, pulmonary hypertension, COPD, restenosis, asthma, neurogenic inflammation metabolic vasculopathies, addiction, schizophrenia, impulsivity, anxiety, stress, depression, neuromuscular function, or diabetes.

9. A process for preparing a compound of formula (I) of claim 1 by
a) alkylating a compound of formula (II): 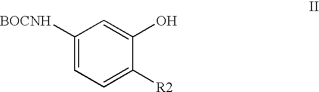
wherein R2 is halogen, CN or methyl;
with a dialkyl amino ethyl chloride;
b) deprotecting to provide a compound of formula (III): 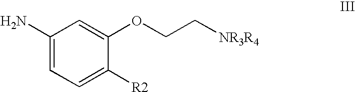
wherein R3 and R4 are independently hydrogen, C₁₋₆ alkyl or benzyl; or with the nitrogen form a pyrrolidine or piperidine ring; and
c) subsequent sulfonylation to provide a compound of formula (I): 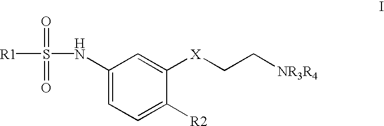
wherein R1, R2, R3, R4 are as defined in claim 1.
